(19) United States
(12) Patent Application Publication
(10) Pub. No.: US 2022/0199220 A1
(43) Pub. Date: Jun. 23, 2022

(54) SYSTEMS AND METHODS OF DIGITALLY-DELIVERED HOME EXERCISE THERAPY

(71) Applicant: LIMBER HEALTH,INC., Bethesda, MD (US)

(72) Inventors: Marc GRUNER, Bethesda, MD (US); Michael GRUNER, Bethesda, MD (US); Nathan HOGABOOM, Bethesda, MD (US)

(21) Appl. No.: 17/645,918

(22) Filed: Dec. 23, 2021

Related U.S. Application Data
(60) Provisional application No. 63/130,277, filed on Dec. 23, 2020.

(51) Int. Cl. G16H 20/30; G16H 40/67

(52) U.S. Cl. CPC G16H 20/30; G16H 40/67

(57) ABSTRACT

A device, method, medium, and system for digitally administered exercise therapy. The device, method, medium, and system may include determining information indicative of a musculoskeletal condition of a patient. The method medium and system may also include determining a likelihood that the patient will have a positive outcome to digitally-delivered exercise therapy based on the information indicative of the musculoskeletal condition of the patient. The method medium and system may also include determining the specific exercises appropriate for the patient's pain and functional ability. Further, the device, method, medium, and system may also including administering a digital exercise therapy regimen to the patient and measuring and tracking the results thereof.

### CROSS-REFERENCE TO RELATED APPLICATIONS

The present application claims the benefit of U.S. Provisional Patent Application No. 63/130,277, filed Dec. 23, 2020, the disclosure of which is incorporated by reference in its entirety herein.

### TECHNICAL FIELD

Aspects of the present disclosure are directed to devices, systems, and methods for digitally-delivered exercise therapy. Specifically, there are three aspects of the present disclosure. First, a software application (“app”) may include a model that may identify a likelihood that a patient will have a positive or negative outcome from digitally derived exercise therapy based on answers to an assessment provided via the application. Second, the app may match specific exercises and exercise therapy programs to the functional disability of the patient. Finally, the app may measure patient outcomes, tracking results to the home-exercise therapy regimen.

### BACKGROUND

Musculoskeletal (MSK) conditions are responsible for more costs to the U.S. healthcare system and cause more disability than any other group of conditions. The annual U.S. cost for MSK care and associated lost wages is estimated at $874 billion (5.7% GDP). Typically the first-line of treatment for MSK conditions is non-operative, and may include home-exercise therapy, physical therapy, orthoses, and/or pharmacological interventions. Early and adherent therapy can improve outcomes, alleviate pain, and reduce total MSK-related healthcare costs (e.g. opioids, injections, surgeries). Unfortunately, conservative treatments for MSK pain are often underutilized, despite evidence that demonstrates lower total healthcare costs and better outcomes with earlier therapy. This effect can especially become a problem for patients living in medically underserved areas or patients with limited access to healthcare due to the lack of insurance coverage or out-of-pocket costs for conservative treatments. Due to access, time, and cost, adherence to therapy regimens remain a significant challenge.

### SUMMARY

The following presents a simplified summary of one or more aspects of the disclosure in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects of the disclosure in a simplified form as a prelude to the more detailed description that is presented later.

According to some aspects, the present disclosure is directed to systems and methods for determining a likelihood that a patient will benefit from a digitally-delivered home-exercise therapy regimen and administering the digitally-delivered regimen to the patient, as well as measuring and tracking results thereof.

To the accomplishment of the foregoing and related ends, the one or more aspects of the disclosure comprise the features hereinafter fully described and particularly pointed out in the claims. The following description and the annexed drawings set forth in detail certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

The novel features believed to be characteristic of aspects described herein are set forth in the appended claims. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative embodiments when read in conjunction with the accompanying drawings, wherein:

FIGS. 1A-1C illustrate a flowchart showing an example method for determining a likelihood of a positive outcome for a patient from a digitally-delivered home-exercise therapy regime, according to an aspect of the disclosure;

FIGS. 2A-2B illustrate a flowchart showing an example method for administering a digitally-delivered home-exercise therapy regime, according to an aspect of the disclosure;

FIG. 3 illustrates an example flowchart showing a summary of a patient interaction with the method 100 and the method 800 according to an example aspect of the disclosure;

FIGS. 4-7 illustrate example graphical user interface (GUI) layouts for a pre-therapeutic and therapeutic processing and treatment, according to an aspect of the disclosure;

FIG. 8 presents an example system diagram of various hardware components and other features for use in accordance with aspects of the disclosure; and

FIG. 9 is a block diagram of various example system components for use in accordance with aspects of the disclosure.

FIG. 10 illustrates a schematic representation of patient movement captured by the system while the patient is completing one or more of the exercises in the video.

FIG. 11 illustrates example graphical user interface (GUI) layouts for pre-therapeutic and therapeutic processing and treatment, according to an aspect of the disclosure.

### DETAILED DESCRIPTION

The following includes definitions of selected terms employed herein. The definitions include various examples and/or forms of components that fall within the scope of a term and that can be used for implementation. The examples are not intended to be limiting.

The term “bus,” as used herein, can refer to an interconnected architecture that is operably connected to transfer data between computer components within a singular or multiple systems. The bus can be a memory bus, a memory controller, a peripheral bus, an external bus, a crossbar switch, and/or a local bus, among others. The bus can also be a vehicle bus that interconnects components inside a vehicle using protocols such as Controller Area Network (CAN), Local Interconnect Network (LIN), among others.

The term “location,” as used herein, can refer to a position of an object in space. A location can be indicated using a coordinate system. For example, a location can be represented as a longitude and latitude. In another aspect, a location can include a height. Moreover, in an example, the location can be relative to an object, such as a device detecting location of another device, and the location can be indicated based on the device detecting the location.

The term “memory,” as used herein, can include volatile memory and/or nonvolatile memory. Non-volatile memory can include, for example, ROM (read only memory), PROM (programmable read only memory), EPROM (erasable PROM) and EEPROM (electrically erasable PROM). Volatile memory can include, for example, RAM (random access memory), synchronous RAM (SRAM), dynamic RAM (DRAM), synchronous DRAM (SDRAM), double data rate SDRAM (DDR SDRAM), and direct RAM bus RAM (DRRAM).

The term “operable connection,” as used herein, can include a connection by which entities are “operably connected,” is one in which signals, physical communications, and/or logical communications can be sent and/or received. An operable connection can include a physical interface, a data interface and/or an electrical interface.

The term “processor,” as used herein, can refer to a device that processes signals and performs general computing and arithmetic functions. Signals processed by the processor can include digital signals, data signals, computer instructions, processor instructions, messages, a bit, a bit stream, or other computing that can be received, transmitted and/or detected. A processor, for example, can include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described herein.

Some of the methods described herein may include a series of questions from a Patient-Reported Outcomes Measurement System (PROMIS®) screening tool. Regarding the PROMIS® screening tool, some beneficial features of PROMIS® measures have greater precision than most conventional measures. Greater precision (less error) enhances power in a less costly way than increasing sample size. In one aspect, PROMIS® measures have a larger range of measurement than most conventional measures, decreasing floor and ceiling effects as a result. Further, PROMIS® measures do all this with fewer items than conventional measures, thereby decreasing respondent burden. When used as computer adaptive tests, PROMIS® measures usually require 4-6 items for precise measurement of health-related constructs. PROMIS® measures provide a common metric: the T-score (mean=50, standard deviation=10). In most cases 50 equals the mean in the U.S. general population. This metric has also been linked to many other conventional measures, and even if other measures are used, it may be possible to report results on the PROMIS® metric, a considerable advantage for ensuring comparability across studies. In another aspect, PROMIS® measures can be administered alongside Neuro-QoL™, ASCQ-Me®, and NIH Toolbox® measures that assess other aspects of health and function.

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

Several aspects of certain systems will now be presented with reference to various example systems and methods. These systems and methods will be described in the following detailed description and illustrated in the accompanying drawings by various blocks, modules, components, circuits, steps, processes, algorithms, etc. (collectively referred to as “elements”). These elements can be implemented using electronic hardware, computer software, or any combination thereof. Whether such elements are implemented as hardware or software depends upon the particular application and design constraints imposed on the overall system.

By way of example, an element, or any portion of an element, or any combination of elements can be implemented with a “processing system” that includes one or more processors. One or more processors in the processing system can execute software. Software shall be construed broadly to mean instructions, instruction sets, code, code segments, program code, programs, subprograms, software modules, applications, software applications, software packages, routines, subroutines, objects, executables, threads of execution, procedures, functions, whether referred to as software, firmware, middleware, microcode, hardware description language, or otherwise.

Accordingly, in one or more aspects, the functions described can be implemented in hardware, software, firmware, or any combination thereof. If implemented in software, the functions can be stored on or encoded as one or more instructions or code on a computer-readable medium. Computer-readable media includes computer storage media. Storage media can be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

In order to help patients effectively self-manage, improve outcomes, and increase adherence to home-exercise therapy regimens, it may be advantageous to administer engaging, functional, and strengthening home-exercise programs that become progressively more challenging over time remotely, for example via digital health applications (“apps”). These digital health apps may be accessed via websites or software applications downloaded onto computing devices, such as laptop computers, desktop computers, tablet computers, and smartphones.

Digital healthcare apps may, among other advantages, be used to predict a patient's response to digitally-delivered exercise therapy and identify patients likely to have a positive outcome to digitally-delivered exercise therapy. Further, digital healthcare apps may help address patient challenges of geographic barriers, costly clinical visits, and travel requirements. Furthermore, digital healthcare apps may help patients self-manage their condition from home, improve outcomes, and increase adherence, given that many patients have access to smartphones and/or other similarly functioning device. Digital healthcare apps may also enable providers to remotely monitor patients.

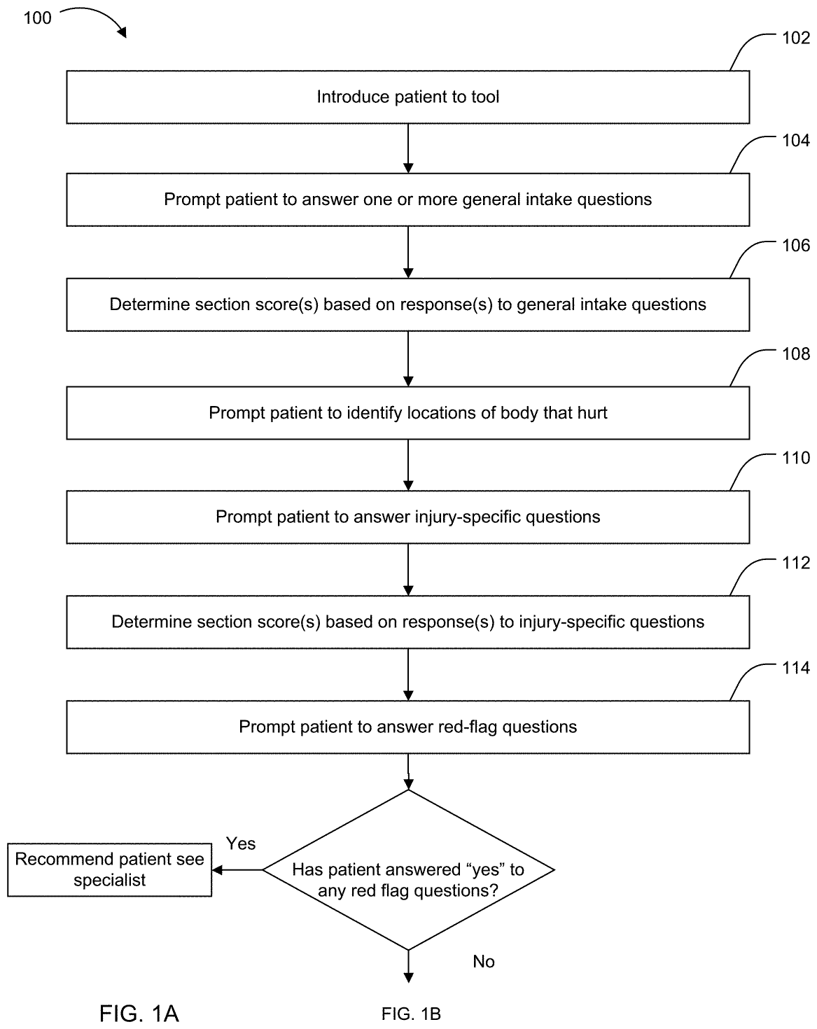

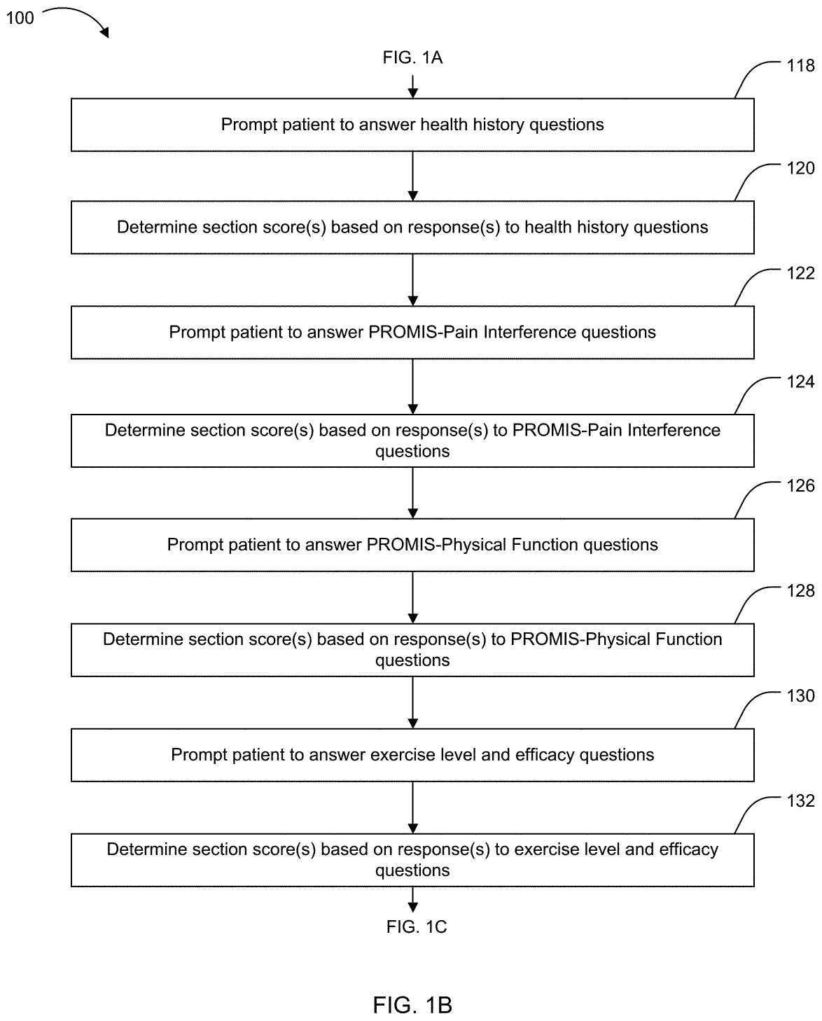

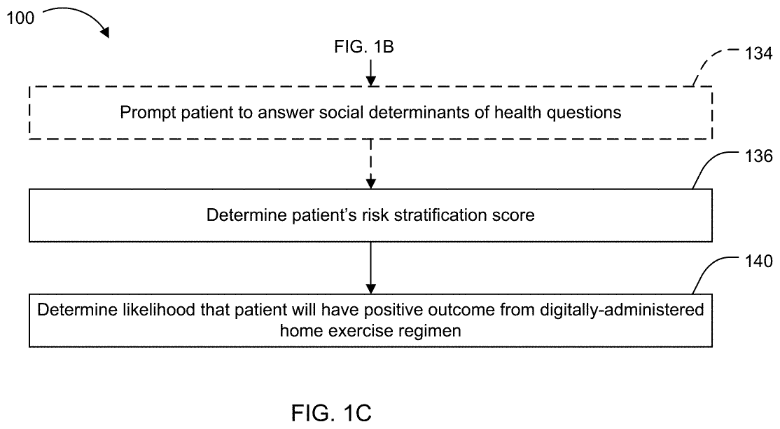

FIGS. 1A-1C illustrate a flowchart showing an example method 100 for determining a likelihood of a positive outcome for a patient from a digitally-delivered exercise therapy regimen. By way of example, the method may be implemented on a computing system such as the computing system 1400.

At block 102, in accordance with one aspect, a patient's healthcare provider (HCP), such as physician, physical therapist, occupational therapist, a patient's insurance provider, and/or a patient's employer, in view of the patient's current symptoms, may make a decision to provide the patient with access to the system for digitally-delivered exercise therapy. In another example, the patient may be able to access the system for digitally-delivered exercise therapy after conducting a network (e.g., Internet or an intranet) search related to the patient's symptoms or history. In accordance with some aspects, the system for digitally-delivered exercise therapy may be or include a tool that the patient is able to use, for example, to monitor the patient's progress through the exercise therapy regimen and/or as improvements in strength, pain, and other symptoms associated with the patient's injury may occur. In accordance with some aspects, the system for digitally-delivered exercise therapy may be or include a tool that the patient's HCP, insurance company, and/or employer is able to use to create longitudinal data and a reference baseline of the patient's musculoskeletal (MSK) wellness, among other purposes.

At block 104, the system 1400 may prompt the patient or other inputter to answer one or more general intake questions or other prompts for response (such questions and other response prompts interchangeably and/or collectively being referred to herein as “questions”). The general intake questions may seek input of such information as the patient's birthday, sex, zip code, height, weight, amount of time sitting per day and so forth. The system 1400 may determine the patient's body mass index (BMI) based on height and weight information provided by the patient. At block 106, the system 1400 may determine section scores based on the answers to the general intake questions. In some aspects of the disclosure, the section scores may range between a value or range of values indicative of a low risk, a value or range of values indicative of a moderate risk, and a value or range of values indicative of a high risk. In accordance with aspects of the disclosure in which the answers include responses to multiple general intake questions associated with a section score, the system 1400 may determine an average section score based on the section scores determined for the patient's answer to each of the general intake questions.

At block 108, the system 1400 may prompt the patient to identify locations of the body in which pain is identified. For example, the system 1400 may display buttons and ask the patient to select the locations of the body having pain, enter the locations of the body that have pain into a text input window, select the portions of the body that have pain on a diagram of the body, and so forth. At block 110, the system 1400 may display one or more injury-specific questions for the locations of the body that have pain. Example locations of the body that have pain include the lower back, shoulder, knee, hip, and neck pain. At block 112, the system may determine section scores based on the answers to the injury-specific questions. In accordance with aspects of the disclosure, the section scores may range between a value or range of values indicative of a low risk, a value or range of values indicative of a moderate risk, and a value or range of values indicative of a high risk. In an aspect of the disclosure in which the answers may include multiple injury-specific questions associated with a section score, the system 1400 may determine an average section score based on the section scores determined for the answer to each of the injury-specific questions.

At block 114, the system 1400 may prompt for answers to one or more injury-specific potential high interest questions (also interchangeably referred to herein as “red flag” questions). The red flag questions may identify, for example, patients who are unlikely to benefit from digitally-delivered exercise therapy or patients who should see a specialist before starting digitally-delivered exercise therapy. The red flag questions may include one or more “yes or no” questions. At block 116, in response to determining that an answer of “yes” has been received for any of the red flag questions, the system 1400 may recommend that the patient see a specialist for evaluation. The patient may not be allowed to continue with the digitally-delivered exercise therapy regimen in response to answering “yes” to one or more of the red flag questions.

If the multiple locations of the body are indicated as having pain, the system 1400 may repeat blocks 110-116 for all of the locations indicated.

At block 118, the system 1400 may prompt for answers to a series of health history questions. In accordance with some aspects of the disclosure, the series of health history questions may include a combination of general health history questions and injury-specific health history questions. In some aspects of the disclosure, the series of health history questions may include general health history questions. At block 120, the system 1400 may determine section scores based on the answers to the injury-specific questions. In accordance with some aspects of the disclosure, the section scores may range between a value or range of values indicative of a low risk, a value or range of values indicative of a moderate risk, and a value or range of values indicative of a high risk. In an aspect of the disclosure in which the answers include multiple health history questions associated with a section score, the system 1400 may determine an average section score based on the section scores determined for the answer to each of the health history questions.

At block 122, the system 1400 may prompt for answers to a series of questions from a Patient-Reported Outcomes Measurement System (PROMIS®) for Pain Interference question bank that are configured to determine self-reported consequences of pain on relevant aspects of a patient's life, which may include the extent to which pain hinders engagement with social, cognitive, emotional, physical, and recreational activities. At block 124, the system 1400 may determine T-scores based on the patient's answers to the PROMIS® Pain Interference questions. The computing system 1400 may determine section scores based on the patient's T-scores. In accordance with an aspect of the disclosure, the section scores may range between a value or range of values indicative of low risk, a value or range of values indicative of moderate risk, and a value or range of values indicative of high risk. In an aspect of the disclosure in which the answers are received to multiple PROMIS® Pain Interference questions associated with a section score, the system 1400 may determine an average section score based on the section scores determined for the answers to each of the PROMIS® Pain Interference questions.

At block 126, the system 1400 may prompt for answers to a series of questions from PROMIS® for Physical Function question bank configured to determine a self-reported ability of a patient to perform various physical activities. The PROMIS® Physical Function questions may be determined based on the locations that the patient has identified as being painful. At block 128, the system 1400 may determine T-scores based on the answers of the patient to the PROMIS® Pain Interference questions. The computing system 1400 may determine section scores based on the T-scores of the patient. In accordance with aspects of the disclosure, the section scores may range between a value or range of values indicative of low risk, a value or range of values indicative of moderate risk, and a value or range of values indicative of high risk. In accordance with aspects of the disclosure, the T-scores of the patient may range from 0-100. In an aspect of the disclosure in which answers are received to multiple PROMIS® Physical Function questions associated with a section score, the system 1400 may determine an average section score based on the section scores determined for the answer of the patient to each of the PROMIS® Physical Functions questions.

At block 130, the system 1400 may prompt the user to answer a series of exercise level and efficacy questions configured to determine, for example, how often and how strenuously the patient exercises. In accordance with aspects of the disclosure, the series of exercise level and efficacy questions may include a combination of general health history questions and injury-specific health history questions. In accordance with aspects of the disclosure, the series exercise level and efficacy questions may include general health history questions. At block 134, the system 1400 may determine section scores based on the answers received for injury-specific questions. In accordance with aspects of the disclosure, the section scores may range between a value or range of values indicative of low risk, a value or range of values indicative of moderate risk, and a value or range of values indicative of high risk.

At optional block 134, the system 1400 may prompt for answers to a series of questions regarding social determinants of health.

At block 136, the system 1400 may determine a risk stratification score based on the section scores the system 1400 determined for one or more of the general intake questions, injury-specific questions, PROMIS® Pain Interference questions, PROMIS® Physical Function questions, exercise level and efficacy questions, and historical health questions, and combinations thereof.

In the example illustrated above, the system 1400 may be configured to determine that a patient having a low risk stratification score between 0-1 is likely to highly benefit from a digitally-delivered home-exercise therapy regimen. The system 1400 may be configured to determine that a patient having a moderate risk stratification score between 1-2 as having a moderate likelihood of benefiting from a digitally-delivered home-exercise therapy regimen. The system 1400 may be configured to determine that a patient having a high risk stratification score between 2-3 as having a lower likelihood of benefiting from a digitally-delivered exercise therapy regimen.

In accordance with aspects of the disclosure, the system 1400 may use machine learning to modify the risk stratification calculation based on patient outcome information. For example, the system may save answers to the questions for each patient involved in the method 100, along with the risk stratification score of the patient, and the actual outcome of the patient for the digitally-delivered exercise therapy regimen. This information may be retained, for example, in a patient information database or other data repository. The system may use the information in the data repository and one or more machine-learning models to modify the Risk Stratification model. Machine-learning models may include, for example, Support Vector Machine (SVM), Decision Tree, Random Forest, AdaBoost, and Multilayer Perceptron (MLP).

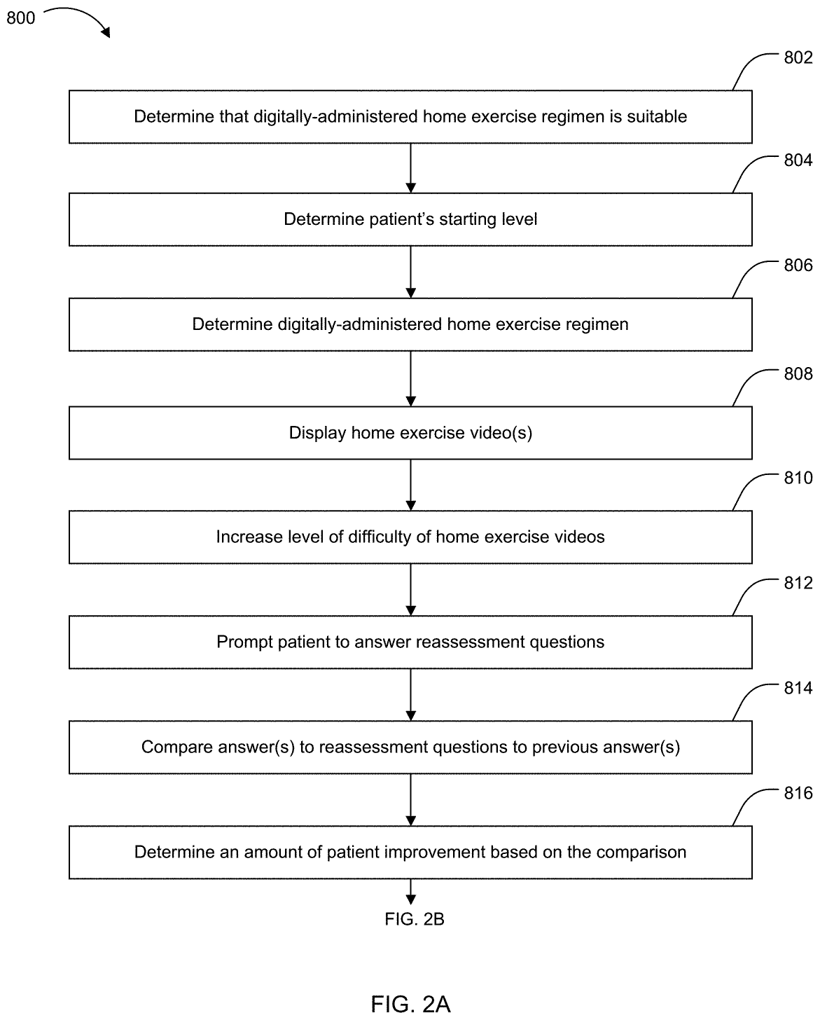

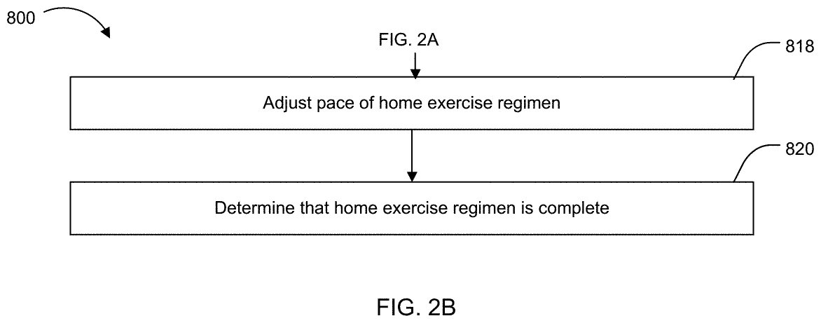

FIGS. 2A-2B illustrate a flowchart showing an example method 800 for administering home-exercise therapy to a patient digitally. The method 100 may start after completion of the method 100 described in FIGS. 1A-1C, for example.

At block 802, the system 1400 may determine that a digitally-delivered exercise therapy regimen is suitable for the patient. For example, the system 1400 may determine the patient will likely have a positive outcome from a digitally-delivered exercise therapy regimen (e.g., based on the method 100). In other implementations, a HCP may recommend the system 1400 to the patient.

At block 804, the system 1400 may determine the starting level for a patient for digitally-delivered home-exercise therapy based on the Risk Stratification of the patient. For example, the system 1400 may determine that patients having a low stratification score should start with a series of more advanced, challenging exercises, “advanced exercises”. The system may determine that patients having a moderate risk stratification score should start with a series of a series of more moderately challenging exercises, “moderate exercises”. The system may determine that patients having a high risk stratification score should start with a series of more basic exercises, “light exercises.”

At block 806, the system 1400 may determine a digitally-delivered exercise therapy regimen for the patient based on the injury of the patient, the pain and function severity of the injury, Risk Stratification score, health, physical capabilities, and goals. For example, the system may provide a more challenging program for a patient with minor pain, average BMI, a goal of returning to competitive sports than for a patient that does not frequently exercise, experiences significant pain, and has a high BMI.

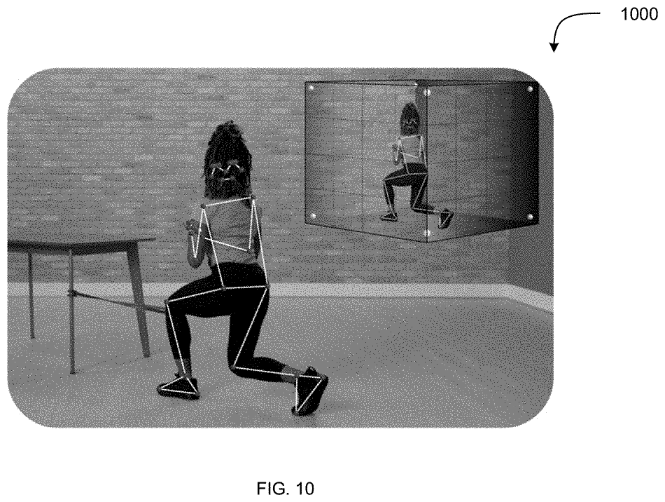

At block 808, the system 1400 may display a exercise therapy video according to the starting level for the patient in response to determining that the patient has initiated a digitally-delivered exercise therapy session. For example, the system 1400 may retrieve a exercise therapy video from an exercise therapy video database based on the digitally-delivered exercise therapy regimen for the patient. The videos may include, for example, detailed, step-by-step instructions of each exercise in the regimen for the patient, tips for improvement, suggestions for monitoring form, etc. The system 1400 may also provide a series of images illustrating the exercises in the exercise therapy regimen for the patient. The system 1400 may also display a list of equipment that the patient is able to use to complete the exercise therapy video (e.g., resistance bands, towels, blankets, weights) The system 1400 may save a completion record for each exercise therapy video displayed to the patient. In accordance with aspects of the disclosure, the system 1400 may prompt the patient to submit a video of the patient completing one or more of the exercises in the video for assessment of the technique of the patient, as proof that the patient completed the exercise therapy video, etc. In some aspects of the disclosure, the system 1400 may capture patient movement data, track patient completion of exercises, and/or provide user feedback to the patient based on data image and/or video data captured by a camera of a patient device such as, for example, a tablet, phone, or computer. FIG. 10 illustrates a schematic representation 1000 of patient movement captured by the system 1400 while the patient is completing one or more of the exercises in the video. In some aspects of the disclosure, the system may receive various monitored, calculated, and/or measured data for the patient, such as motion tracking data from the patient's portable computing device or a smart accessory device that includes an accelerometer. For example, the system 1400 may analyze the motion tracking data to identify information indicative of a patient's motion to determine a range of motion of the patient, analyze a technique of the patient, and/or record patient compliance with the exercise therapy video based on the data, and so forth.

At block 810, the system 1400 may increase the level of difficulty of exercise therapy videos provided to the patient, in accordance with aspects of the present disclosure. Further, for example, the system 1400 may increase the level of exercise therapy videos based on a number of times that the patient has completed exercise therapy videos at a particular level.

At block 812, the system 1400 may prompt for responses to one or more reassessment questions after a predefined time period. In accordance with aspects of the disclosure, the predefined time period may be 4 weeks since the start of exercise therapy, 8 weeks since the start of exercise therapy, 12 weeks after the start of exercise therapy, 6 months after the start of exercise therapy, 1 year after the start of exercise therapy, and/or 2 years after the start of exercise therapy. The reassessment questions may include PROMIS® Pain Interference and PROMIS® Physical Function questions.

At block 814, the system 1400 may compare the answers received to the PROMIS® Pain Interference and PROMIS® Physical Function questions to the previous answers received to the PROMIS® Pain Interference and PROMIS® Physical Function questions. The system may compare the answers in a first reassessment to the answers to the PROMIS® Pain and Interference and PROMIS® Physical Function questions in the method 100. In subsequent reassessments, the system may compare the answers in a current reassessment to the answers to the PROMIS® Pain and Interference and PROMIS® Physical Function questions in a previous reassessment. At block 816, the system 1400 may determine an amount of improvement based on the comparison.

At block 818, the system may adjust the progress of the patient through the exercise therapy regimen based on the amount of improvement of the patient. For example, in response to determining that the amount of improvement of the patient is below a predefined threshold, the system 1400 may slow down the rate at which the level of the exercise therapy videos for the patient is increased. In response to determining that the amount of improvement of the patient is above a predefined threshold, the system 1400 may increase the rate at which the level of the video for the patient is increased or maintain the rate at which the level of the level video of the patient is increased.

At block 820, the system 1400 may determine that the home-exercise therapy regimen for the patient is complete. The system 1400 may determine that the regimen for the patient is complete, for example, based on an amount of time the patient spent in the exercise therapy regimen, a level of exercise therapy videos completed by the patient, an amount of improvement of the patient based on PROMIS® Pain Interference and PROMIS® Physical Function questions, and so forth.

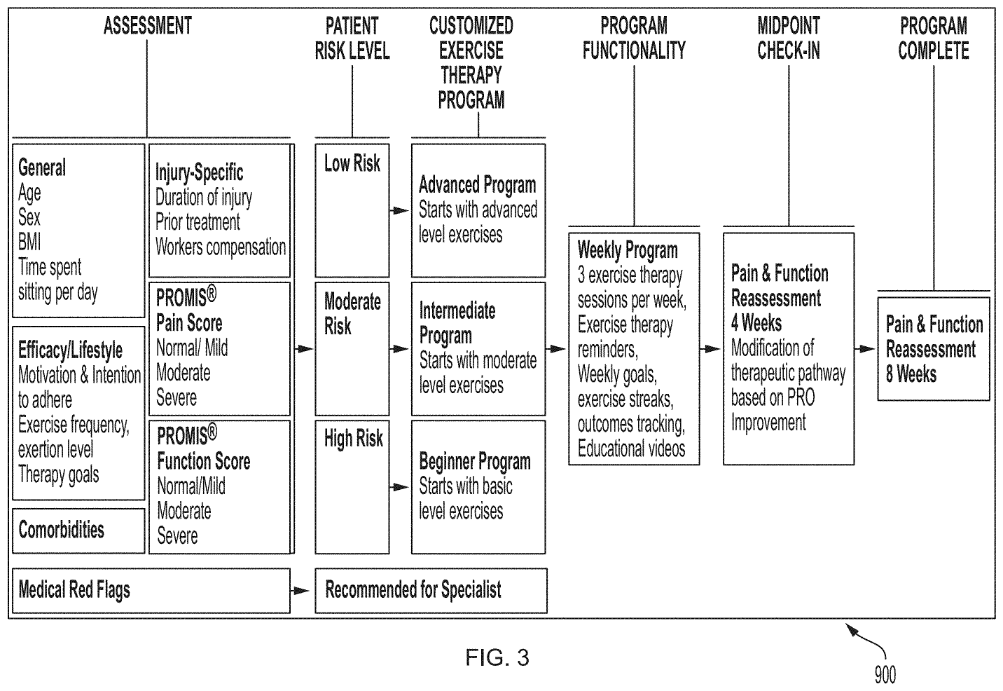

FIG. 3 illustrates a simple overview representative block diagram 900 of patient interaction with the system 1400 that includes elements of method 100 and method 800 according to an example embodiment.

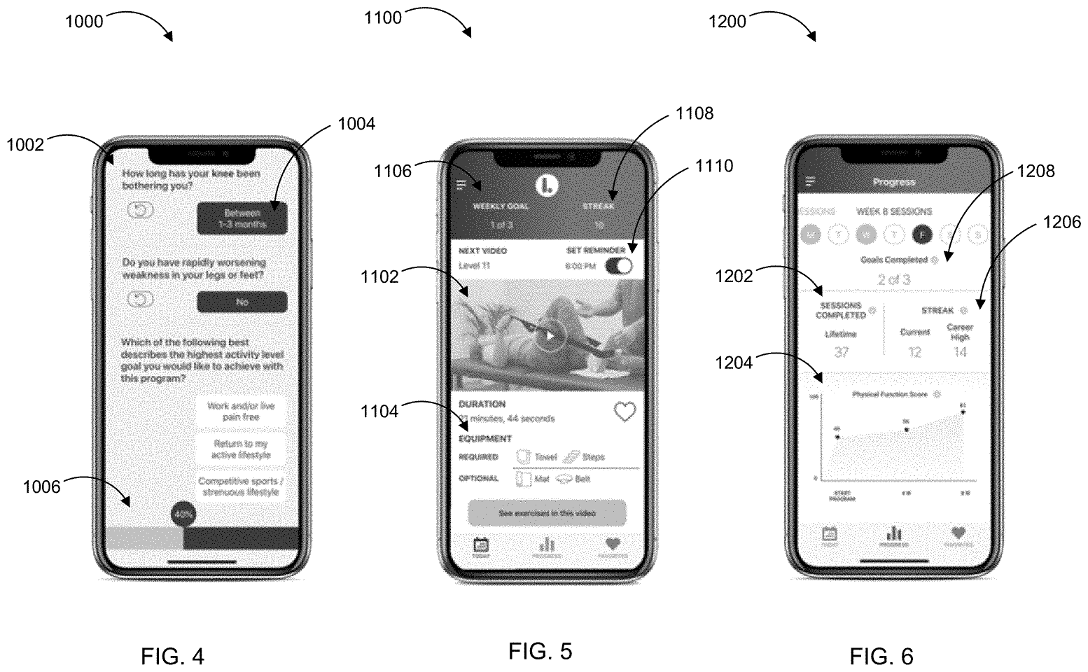

FIGS. 4-7 show example GUI screens for implementing aspects of the present disclosure. FIG. 4 illustrates an example GUI screen 1000 that may be displayed to a patient during the method 100 described in FIGS. 1A-1C described above. The example display 1000 may include questions 1002 prompting for input of patient data, buttons 1004 configured to receive a response to the questions 1002, and a status bar 1006 indicating progress through the method 100. FIGS. 5, 6, and 7 show example GUI screens 1100, 1200, and 1300, respectively, that may be displayed to a patient during various portions of the method 800 described in FIGS. 2A-2B above.

The screen 1100 may be displayed, for example, to a patient when a patient initiates a digitally-delivered exercise therapy session. The screen 1100 may include an exercise therapy video 1102 and a list of equipment 1104 required or recommended for use with the exercise therapy video 1102. The screen 1100 may also include information intended to motivate a patient to adhere to the exercise therapy regimen, including, for example, a weekly goal indicator 1106, a streak indicator 1108, and a slider bar 1110 configured to enable the setting of reminders.

The screen 1200 may be displayed to summarize the progress of the patient during the digitally-delivered exercise therapy regimen. The screen 1200 may include indicators of progress of a patient through the digitally-delivered exercise therapy regimen, including an indication of exercise therapy sessions completed 1202 and a graphic 1204 indicative of the improvement of the patient during the digitally-delivered exercise therapy regimen, a streak indicator 1206, and a goal indicator 1208. The GUI screens may be presented, for example, on a mobile device, computer, or tablet, among other devices.

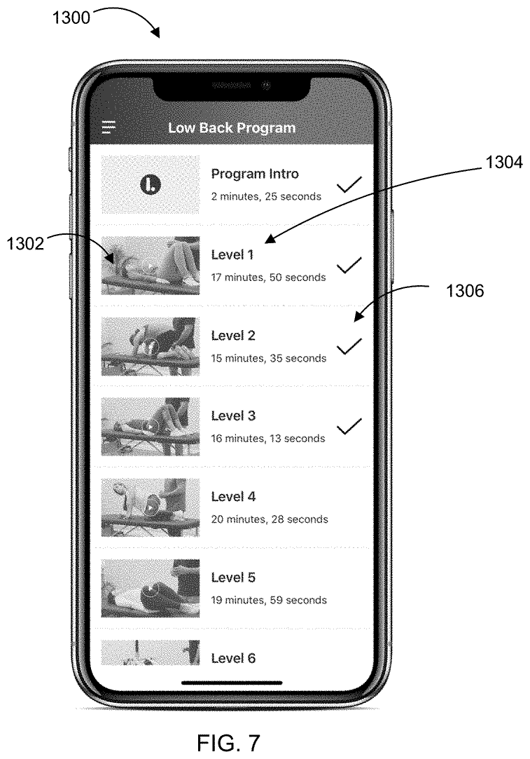

The screen 1300 may be displayed to summarize an exercise therapy regimen of the patient. The screen 1300 may show thumbnails 1302 of the exercise therapy videos of the exercise therapy regimen, the 1304 levels of the exercise therapy videos, and status indicators 1306. In the example illustrated in FIG. 7, the status indicators may be check marks indicating completed videos. Other status indicators may be used in other aspects of the disclosure.

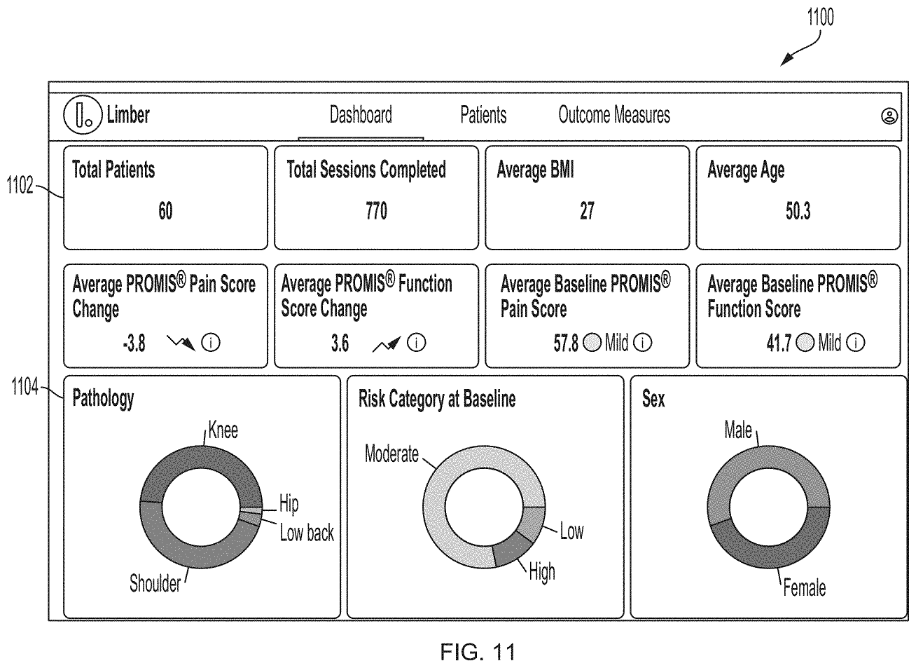

FIG. 11 illustrates an example GUI screen 1100 that may be displayed to a HCP, an insurance company employee, and/or an employer during the method 800 described in FIGS. 2A-2B described above. In some embodiments, the example screen 1100 may display information indicative of the progress of an HCP's patients. For example, the screen 1100 may display buttons indicative aggregated information about a HCP's patients as one or more buttons 1102. The buttons may include information such as a total number of patients, a total number of exercise therapy sessions completed, an average patient BMI, and average patient age, change in PROMIS® Pain Interference scores, change in PROMIS® Physical Function scores, average PROMIS® Pain interference scores, average PROMIS® Physical Function scores, and so forth. In some aspects of the disclosure, selecting one of the buttons 1102 may cause the GUI screen to display a detailed view of the information on a per-patient basis. The GUI screen 1100 may include graphical summary data 1104. The graphical summary data 1104 may include locations that have pain, risk levels, sex of the patients, and so forth. In other aspects of the disclosure, the example screen 1100 may display a record of exercise therapy videos completed by each patient, initial assessment and reassessment results for each patient, patient risk stratification scores, and other patient data collected by the system 1400.

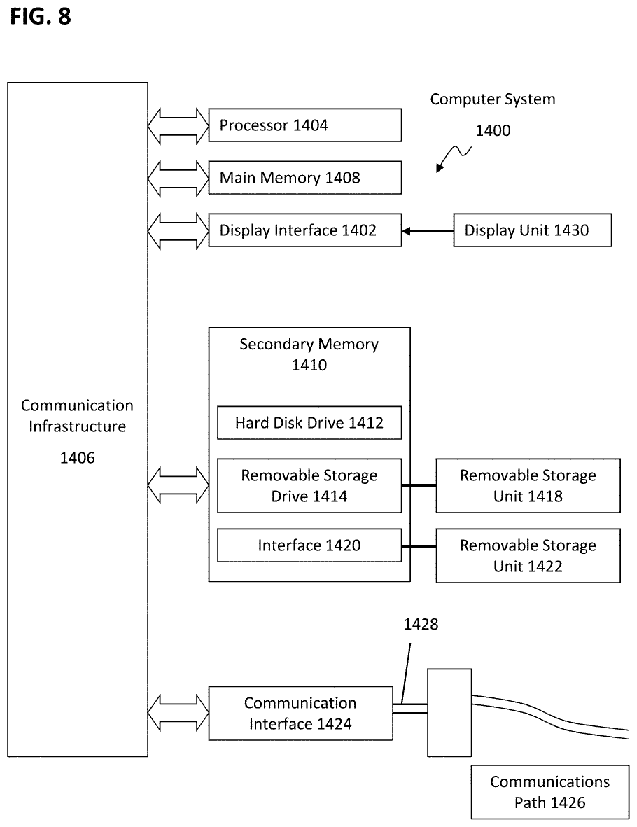

Aspects of the present disclosure may be implemented using hardware, software, or a combination thereof and can be implemented in one or more computer systems or other processing systems. In one aspect, the disclosure is directed toward one or more computer systems capable of carrying out the functionality described herein. An example of such a computer system 14 is shown in FIG. 8.

FIG. 8 presents an example system diagram of various hardware components and other features, for use in accordance with an aspect of the present disclosure. Aspects of the present disclosure can be implemented using hardware, software, or a combination thereof and can be implemented in one or more computer systems or other processing systems. In one example variation, aspects described herein can be directed toward one or more computer systems capable of carrying out the functionality described herein. An example of such a computer system 1400 is shown in FIG. 8.

Computer system 1400 includes one or more processors, such as processor 1404. The processor 1404 is connected to a communication infrastructure 1406 (e.g., a communications bus, cross-over bar, or network). In one example, processor 1420 can include processor 1404. Various software aspects are described in terms of this example computer system. After reading this description, it will become apparent to a person skilled in the relevant art(s) how to implement aspects described herein using other computer systems and/or architectures.

Computer system 1400 can include a display interface 1402 that forwards graphics, text, and other data from the communication infrastructure 1406 (or from a frame buffer not shown) for display on a display unit 1430. Computer system 1400 also includes a main memory 1408, preferably random access memory (RAM), and can also include a secondary memory 1410. The secondary memory 1410 can include, for example, a hard disk drive 1412 and/or a removable storage drive 1414, representing a floppy disk drive, a magnetic tape drive, an optical disk drive, etc. The removable storage drive 1414 reads from and/or writes to a removable storage unit 1418 in a well-known manner. Removable storage unit 1418, represents a floppy disk, magnetic tape, optical disk, etc., which is read by and written to removable storage drive 1414. As will be appreciated, the removable storage unit 1418 includes a computer usable storage medium having stored therein computer software and/or data.

In alternative aspects, secondary memory 1410 can include other similar devices for allowing computer programs or other instructions to be loaded into computer system 1400. Such devices can include, for example, a removable storage unit 1422 and an interface 1420. Examples of such can include a program cartridge and cartridge interface (such as that found in video game devices), a removable memory chip (such as an erasable programmable read only memory (EPROM), or programmable read only memory (PROM)) and associated socket, and other removable storage units 1422 and interfaces 1420, which allow software and data to be transferred from the removable storage unit 1422 to computer system 1400.

Computer system 1400 can also include a communications interface 1424. Communications interface 1424 allows software and data to be transferred between computer system 1400 and external devices. Examples of communications interface 1424 can include a modem, a network interface (such as an Ethernet card), a communications port, a Personal Computer Memory Card International Association (PCMCIA) slot and card, etc. Software and data transferred via communications interface 324 are in the form of signals 328, which can be electronic, electromagnetic, optical or other signals capable of being received by communications interface 1424. These signals 1428 are provided to communications interface 1424 via a communications path (e.g., channel) 1426. This path 1426 carries signals 1428 and can be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 1480, a hard disk installed in hard disk drive 1470, and signals 1428. These computer program products provide software to the computer system 1400. Aspects described herein can be directed to such computer program products.

Computer programs (also referred to as computer control logic) are stored in main memory 1408 and/or secondary memory 1410. Computer programs can also be received via communications interface 1424. Such computer programs, when executed, enable the computer system 1400 to perform various features in accordance with aspects described herein. In particular, the computer programs, when executed, enable the processor 1404 to perform such features. Accordingly, such computer programs represent controllers of the computer system 1400.

In variations where aspects described herein are implemented using software, the software can be stored in a computer program product and loaded into computer system 1400 using removable storage drive 1414, hard disk drive 1412, or communications interface 1420. The control logic (software), when executed by the processor 1404, causes the processor 1404 to perform the functions in accordance with aspects described herein as described herein. In another variation, aspects are implemented primarily in hardware using, for example, hardware components, such as application specific integrated circuits (ASICs). Implementation of the hardware state machine so as to perform the functions described herein will be apparent to persons skilled in the relevant art(s).

In yet another example variation, aspects described herein are implemented using a combination of both hardware and software.

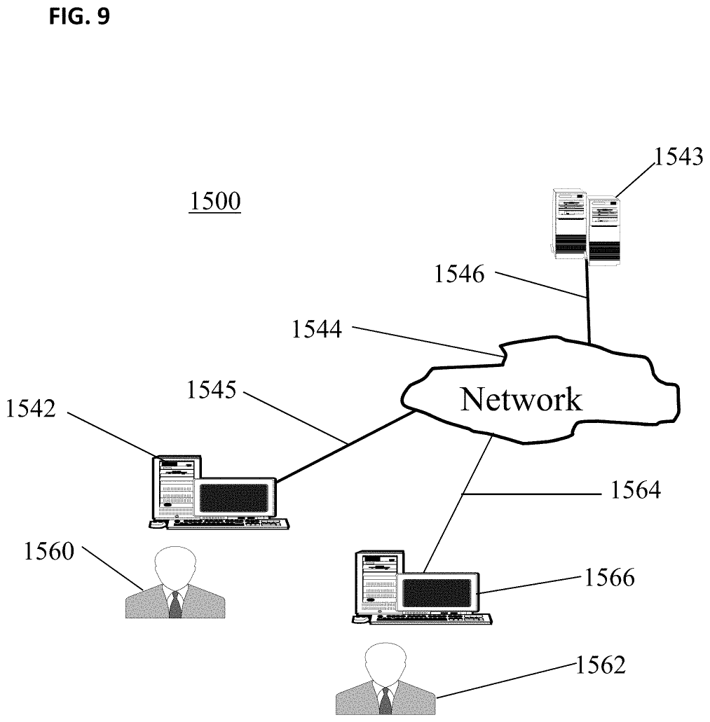

FIG. 9 is a block diagram of various example system components, in accordance with an aspect. FIG. 9 shows a communication system 1500 usable in accordance with various aspects described herein. The communication system 1500 includes one or more accessors 1560, 1562 (also referred to interchangeably herein as one or more “users” or “patients”) and one or more terminals 1542, 1566. For example, terminals 1542, 1566 may include remote device 104, and/or the like. In one aspect, data for use in accordance with aspects described herein is, for example, input and/or accessed by accessors 1506, 1562 via terminals 1542, 1566, such as personal computers (PCs), minicomputers, mainframe computers, microcomputers, telephonic devices, or wireless devices, such as personal digital assistants (“PDAs”) or a hand-held wireless devices coupled to a server 1543, such as a PC, minicomputer, mainframe computer, microcomputer, or other device having a processor and a repository for data and/or connection to a repository for data, via, for example, a network 1544, such as the Internet or an intranet, and couplings 1545, 1546, 1564. The couplings 1545, 1546, 1564 include, for example, wired, wireless, or fiber optic links. In another example variation, the method and system in accordance with aspects described herein operate in a stand-alone environment, such as on a single terminal.

The aspects discussed herein can also be described and implemented in the context of computer-readable storage medium storing computer-executable instructions. Computer-readable storage media includes computer storage media and communication media. For example, flash memory drives, digital versatile discs (DVDs), compact discs (CDs), floppy disks, and tape cassettes. Computer-readable storage media can include volatile and nonvolatile, removable and non-removable media implemented in any method or technology for storage of information such as computer readable instructions, data structures, modules or other data.

It will be appreciated that various implementations of the above-disclosed and other features and functions, or alternatives or varieties thereof, can be desirably combined into many other different systems or applications. Also that various presently unforeseen or unanticipated alternatives, modifications, variations, or improvements therein can be subsequently made by those skilled in the art which are also intended to be encompassed by the following claims.

What is claimed is:

1. A computer-implemented method for generating and administering a digital-delivered exercise therapy regimen to a patient, the method comprising:
determining information indicative of a musculoskeletal condition of a patient;
determining a likelihood that the patient will have a positive or negative outcome to digitally-delivered exercise therapy based on the information indicative of the musculoskeletal condition of the patient;
in response to determining that the patient is likely to have a positive outcome to digitally-delivered exercise therapy:
determining the digitally-delivered exercise therapy regimen matched appropriately to a pain and functional ability of the patient; and
administering the digitally-delivered exercise therapy regimen to the patient.

2. The computer-implemented method of claim 1, wherein the determining the likelihood that the patient will have a positive or negative outcome to digitally-delivered exercise therapy includes one or more of prompting the patient to answer general intake questions, identify locations of a body that have pain, answer health history questions, answer questions related to Patient-Reported Outcomes Measurement System (PROMIS®) for Pain Interference, answer questions related to PROMIS® Physical Function, answer questions related to exercise level and efficacy, answer questions related to social determinants of health, and combinations thereof.

3. The computer-implemented method of claim 2, further comprising determining a risk stratification score based on one or more of the answers to the general intake questions, identified locations of the body that have pain, answers to the health history questions, answers to the Patient-Reported Outcomes Measurement System (PROMIS®) for Pain Interference, answers to the questions related to PROMIS® Physical Function, answers to the questions related to exercise level and efficacy, answers to the questions related to social determinants of health, and combinations thereof; and
determining the likelihood that the patient will have a positive or negative outcome to digitally-delivered exercise therapy based on the risk stratification score.

4. The computer-implemented method of claim 3, wherein the digitally-delivered exercise therapy regimen is determined based on the risk stratification score.

5. The computer-implemented method of claim 1, wherein administering the digitally-delivered exercise therapy regimen includes retrieving an exercise video from an exercise therapy video database or retrieving a series of images illustrating exercises in the digitally-delivered exercise therapy regimen; and
displaying the exercise video or the series of images illustrating exercises in the digitally-delivered exercise therapy regimen to the patient.

6. The computer-implemented method of claim 1, further comprising one or more of capturing patient movement data, tracking patient completion of the digitally-delivered exercise therapy regimen, and/or providing feedback to the patient based on data image and/or video data captured by a camera.

7. The computer-implemented method of claim 1, further comprising receiving motion tracking data to identify information indicative of a patient's motion to determine a range of motion of the patient, analyze a technique of the patient, and/or record patient compliance with an exercise therapy video based on the data, and combinations thereof.

8. The computer-implemented method of claim 7, wherein the motion tracking data is received from an accelerometer.

9. The computer-implemented method of claim 1, further comprising increasing a level of difficulty of the digitally-delivered exercise therapy regimen based on a number of times that the patient has completed exercise therapy videos at a particular level.

10. The computer-implemented method of claim 1, further comprising prompting the patient to answer one or more reassessment questions after a predefined time period.

11. The computer-implemented method of claim 10, further comprising determining an amount of patient improvement based on the answers to the reassessment questions; and
adjusting progress of the patient through the digitally-delivered exercise therapy regimen based on the amount of patient improvement.

12. A system for administering generating and administering a digital home-exercise therapy regimen to a patient, comprising:
a memory;
at least one processor coupled to the memory and configured to:
determine information indicative of a musculoskeletal condition of a patient;
determine a likelihood that the patient will have a positive or negative outcome to digitally-delivered exercise therapy based on the information indicative of the musculoskeletal condition of the patient;
in response to determining that the patient is likely to have a positive outcome to digitally-delivered exercise therapy:
determine a digital home-exercise therapy regimen matched appropriately to a pain and functional ability of the patient; and
administer the digital home-exercise therapy regimen to the patient.

13. The system of claim 12, wherein the processor is configured to prompt the patient to answer general intake questions, identify locations of a body that have pain, answer health history questions, answer questions related to Patient-Reported Outcomes Measurement System (PROMIS®) for Pain Interference, answer questions related to PROMIS® Physical Function, answer questions related to exercise level and efficacy, answer questions related to social determinants of health, and combinations thereof.

14. The system of claim 13, wherein the processor is configured to determine a risk stratification score based on one or more of the answers to the general intake questions, identified locations of the body that have pain, answers to the health history questions, answers to the Patient-Reported Outcomes Measurement System (PROMIS®) for Pain Interference, answers to the questions related to PROMIS® Physical Function, answers to the questions related to exercise level and efficacy, answers to the questions related to social determinants of health, and combinations thereof; and
determine the likelihood that the patient will have a positive or negative outcome to digitally-delivered exercise therapy based on the risk stratification score.

15. The system of claim 12, wherein administering the digitally-delivered exercise therapy regimen includes retrieving an exercise video from an exercise therapy video database or retrieving a series of images illustrating exercises in the digitally-delivered exercise therapy regimen; and
displaying the exercise video or the series of images illustrating exercises in the digitally-delivered exercise therapy regimen to the patient.

16. The system of claim 12, wherein the processor is configured determine patient movement data, track patient completion of the digitally-delivered exercise therapy regimen, and/or provide feedback to the patient based on data image and/or video data captured by a camera.

17. The system of claim 12, wherein the processor is configured to receive motion tracking data to identify information indicative of a patient's motion to determine a range of motion of the patient, analyze a technique of the patient, and/or record patient compliance with the digital home-exercise therapy video based on the data, and combinations thereof.

18. The system of claim 12, wherein the processor is configured to increase a level of difficulty of the digital home-exercise therapy regimen based on a number of times that the patient has completed exercise therapy videos at a particular level.

19. The system of claim 12, wherein the processor is configured to prompt the patient to answer one or more reassessment questions after a predefined time period.

20. The system of claim 19, wherein the processor is configured to:
determine an amount of patient improvement based on the answers to the reassessment questions; and
adjust progress of the patient through the digitally-delivered exercise therapy regimen based on the amount of patient improvement
